(19) United States
(12) Patent Application Publication
(10) Pub. No.: US 2012/0129949 A1
(43) Pub. Date: May 24, 2012

(54) Use of Anthracene Derivatives as Anti-Infectives

(75) Inventors: Alexander Pretsch, Grossmugl (AT); Peter Proksch, Julich (DE); Abdessamad Debbab, Dusseldorf (DE)

(73) Assignee: SEALIFE PHARMA GMBH, Tulln (AT)

(21) Appl. No.: 13/375,059

(22) PCT Filed: May 31, 2010
(86) PCT No.: PCT/AT2010/000189

§ 371 Date: Feb. 7, 2012

(30) Foreign Application Priority Data
May 29, 2009 (AT) A 843/09

(51) Int. Cl. A61K 31/122; A61P 31/04; A61P 31/12; C12P 15/00

(52) U.S. Cl. 514/680; 435/127

(57) ABSTRACT

The present invention relates to the use of the following anthracene derivatives as anti-infectives, preferably against multiply drug resistant pathogens:
(2R,3S,10R)-2,3,5,10-tetrahydroxy-6-methoxy-3-methyl-1,2,3,4,4a,9a-hexahydro-9H,10H-anthracen-9-one (tetrahydroaltersolanol B),
(1R,2R,3R,4aS,9aR,10R)-1,2,3,5,10-pentahydroxy-7-methoxy-2-methyl-1,2,3,4,4a,9a-hexahydro-9H,10H-anthracen-9-one (altersolanol K),
(2R,3R,4R,4aS,9aS,10R)-2,3,4,8,10-pentahydroxy-6-methoxy-3-methyl-1,2,3,4,4a,9a-hexahydro-9H,10H-anthracen-9-one (altersolanol L),
8-(1,7-dihydroxy-6-methyl-3-methoxy-9,10-dioxo-9H,10H-anthracen-2-yl)-(1S,2S,3R,4S)-1,2,3,4,5-pentahydroxy-7-methoxy-2-methyl-1,2,3,4-tetrahydro-9H,10H-anthracen-9,10-dione (atropisomers alterporriol G and H); as well as
(2R,3S,4aS,9aS,10R)-2,3,5,8,10-pentahydroxy-6-methoxy-3-methyl-1,2,3,4,4a,9a-hexahydro-9H,10H-anthracen-9-one (8-hydroxytetrahydroaltersolanol B).

The present invention relates to the use of anthracene derivatives as anti-infectives, especially against multiply drug-resistant pathogens, as well as to a method for their production.

### STATE OF THE ART

In the late 1960s and early 1970s, it was assumed, due to the highly successful use of anti-microbial drugs, that infectious diseases would not constitute any danger any more. This, however, proved to be a misconception, all the more so as, forty years later, microbes are constituting a greater threat than ever before. For this reason, there is an urgent need for new anti-microbial agents. These days, infectious diseases constitute the third most frequent cause of death in the US, and the second most frequent cause of death at a global level. Ineffective anti-microbial drugs are responsible for most of these cases, and the resistance of certain bacteria and fungi to these agents confronts our society with a serious problem. According to statistical data from the US, the majority of infections contracted in hospitals (the so called nosocomial infections) is caused by a small number of bacteria species, namely by Enterococcus faecium, Staphylococcus aureus, Klebsiella pneumoniae, Acinetobacter baumanii, Pseudomonas aeruginosa, and Enterobacter sp., which, based on their starting letters, are collectively referred to as “ESKAPE” pathogens (Boucher et al., “IDSA Report on Development Pipeline”, CID 2009:48, Infectious Disease Society of America, 2009-01-01). At the same time, this refers to the fact that these resistant pathogens escape the effect of anti-bacterial drugs, particularly as a resistance to antibiotics, at the molecular level, is nothing else than the ability acquired by a microorganism to resist the growth inhibiting or bactericidal action of an anti-microbial substance. This means that the substance becomes clinically ineffective. A “methicillin-resistant Staphylococcus aureus” (abbreviated as MRSA; this abbreviation being, however, also used in a more general sense for “multi-resistant Staphylococcus aureus”) means that the use of β-lactams is ineffective in the therapy of S. aureus, while glycopeptides often still have an effect. It is, thus, absolutely necessary to test the action new anti-infectives have on multi-resistant strains, as, even though they are effect against species and strains which are sensitive to them, this does not automatically mean that they are also effective against multi-resistant bacteria, just like an agent against Gram-positive bacteria is not automatically effective against Gram-negative bacteria and vice versa (see Boucher et al., supra, etc.)

Apart from strategies against bacteria and fungi, we currently also lack effective strategies against respiratory viruses. In most cases, it is just the symptoms which are cured, without fighting the virus itself. A solution for the future could consist mainly in combinations of active agents.

In the past, numerous substances for fighting the proliferation of microorganisms were found in endophytic fungi. In a number of cases, these substances have very good anti-bacterial, anti-fungal, and anti-viral activities and may be used for multiple applications (G. A. Strobel, Crit. Rev. Biotechnol. 22, 315-333 (2002)). Due to the fact that research in this field was carried out mainly at an academic level and did not directly aim at the development of new active agents, there are hardly any suitable drugs available on the market today. Especially screening against resistant microbes was neglected in the past, so that only a few substances against drug-resistant microbes have been tested. The situation is even worse in the case of respiratory viruses; so far, hardly any substances effective against respiratory viruses have been screened.

Already in 1969, A. Stoessl, Can. J. Chem. 47, 767 (1969) described the isolation of a new metabolic pigment from Alternaria solani, a mold fungus causing a disease called early blight in potatoes. This pigment was named altersolanol A, and its structure was identified to look as follows:

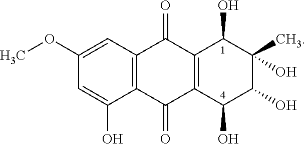

Thus, the chemical name of this anthraquinone is 7-methoxy-2-methyl-(1R,2S,3R,4S)-1,2,3,4-tetrahydro-1,2,3,4,5-pentahydroxy-anthracene-9,10-dione.

Subsequently, this pigment as well as several isomers and derivatives thereof were detected in further Alternaria species (such as Alternaria porn) and in some other fungal genera (see, for example, R. Suemitsu et al., Agric. Biol. Chem. 45(10), 2363-2364 (1981)). The majority of the isomers and derivatives corresponds to one of the following general formulae (1) and (2):

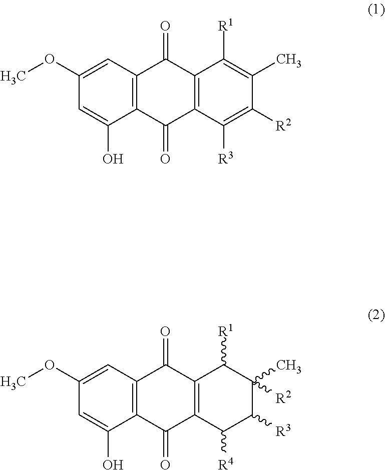

wherein R¹ to R⁴ may each represent H or OH, which, in the case of OH in formula (2), results in one chiral carbon atom, which may be in R- or S-configuration.

Subsequently, dimers, i.e. bisanthraquinones, were discovered as of metabolites, first in Alternaria porri, another representative of the genus Alternaria, which, amongst others, causes the purple blotch disease in onions, which is why these dimers were named alterporriols. They, for example, correspond to the following formula 3:

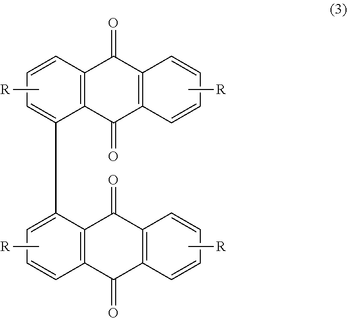

wherein the possible number of diverse substitution and hydration patterns of the aromatic rings is similar to that in “monomeric” altersolanols. Due to the restricted rotational freedom about the axis of the chemical bond between the anthraquinone cores, a high number of alterporriol derivatives exist in the form of two atropisomers.

There are reports on some compounds referred to as altersolanols and alterporriols concerning their effectiveness against certain microorganisms, while others have been described as not having any anti-infective effect. Aly et al., Phytochemistry 69, 1716-1725 (2009), for example, examined bioactive metabolites of endophytic fungi from the genus Ampelomyces as well as their anti-infective action. Among other things, they found out that altersolanol J, the alterporriols D and E (atropisomers) as well as the compound ampelanol, which is closely related to altersolanols, do not show any activity against bacteria and fungi, whereas altersolanol A proved to be the most efficient of the tested substances. Moreover, Okamura et al., Phytochemistry 42(1), 77-80 (1996), for example, do not disclose any effect of tetrahydroaltersolanol B against Gram-positive bacteria and the Gram-negative species Pseudomonas aeruginosa. Yagi et al., Phytochemistry 34(4), 1005-1009 (1993) report on the anti-microbial activity of the altersolanols A, B, C, and E, whereas the altersolanols D, E and F proved to be completely ineffective in the same test. US application No. 2007/258913, on the other hand, discloses the atropisomeric altersolanol D and E as compounds which are suitable for preventing the formation of a biofilm in the oral cavity, only in a very general way, though, and without listing any specific data concerning their effectiveness.

Thus, it is impossible to predict whether a specific altersolanol or alterporriol isomer or derivative will show an anti-infective effect or not, and still less against which genera or species of microorganisms.

Against this backdrop, the aim of the present invention consisted in the identification, isolation, and production of new substances to be used as anti-infective agents in pharmaceutical compositions, particularly of compounds showing an activity against multi-resistant (“multiply drug resistant”, MDR) pathogens.

In the course of their research work, the inventors were able to identify several substances—some of them having so far unpublished structures, i.e. new chemical compounds—which show good activities against microorganisms, but also against respiratory viruses, especially against MDR pathogens. While the parallel, simultaneously filed Austrian Patent Application with the Application No. A 842/09 provides two novel compounds, one altersolanol and one alterporriol derivative, the present invention relates to the use of several compounds, the structure of which has already been known from literature, but which have an effect which has been unknown so far.

### DISCLOSURE OF THE INVENTION

More specifically, the inventors have found out that the following anthracene derivatives sometimes show excellent activities against various bacteria, fungi, and viruses; for this reason, a first aspect of the present invention relates to the use of these derivatives as anti-infectives:

### a) (2R,3S,10R)-2,3,5,10-tetrahydroxy-6-methoxy-3-methyl-1,2,3,4,4a,9a-hexahydro-9H,10H-anthracen-9-one (tetrahydroaltersolanol B) of formula (4)

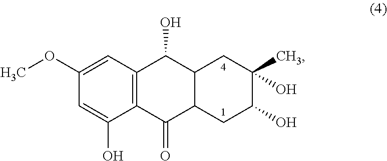

### b) (1R,2R,3R,4aS,9aR,10R)-1,2,3,5,10-pentahydroxy-7-methoxy-2-methyl-1,2,3,4,4a,9a-hexahydro-9H,10H-anthracen-9-one (altersolanol K) of formula (5)

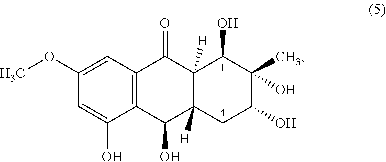

### c) (2R,3R,4R,4aS,9aS,10R)-2,3,4,8,10-pentahydroxy-6-methoxy-3-methyl-1,2,3,4,4a,9a-hexahydro-9H,10H-anthracen-9-one (altersolanol L) of formula (6)

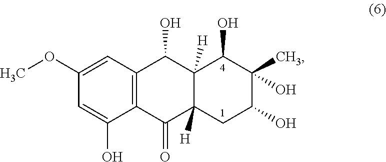

and

### d) 8-(1,7-dihydroxy-6-methyl-3-methoxy-9,10-dioxo-9H,10H-anthracen-2-yl)-(1S,2S,3R,4S)-1,2,3,4,5-pentahydroxy-7-methoxy-2-methyl-1,2,3,4-tetrahydro-9H,10H-anthracen-9,10-dione (atropisomers alterporriol G and H) of formula (7)

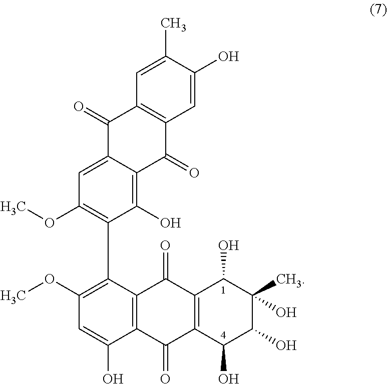

These anthracene derivatives are preferably used against multiply drug resistant (MDR) pathogens, as their efficiency in this respect ranges from very good to excellent. The structures of these five substances were already published in the past, but hardly anything is known about their effects.

As mentioned above, Okamura et al. (supra) even describe compound (4), tetra-hydroaltersolanol B, as inefficient against Gram-positive bacteria and the Gram-negative species Pseudomonas aeruginosa. No activity tests have been disclosed for the compounds (5), (6), and (7), i.e. altersolanol K, altersolanol L, and the atropisomers alterporriol G and H. All of them were first isolated from Stemphylium globuliferum by co-inventors of the present application object, as published on the website of the Journal of Natural Products on 9 Mar. 2009 (printed edition in production).

The invention further relates to the use of (2R,3S,4aS,9aS,10R)-2,3,5,8,10-penta-hydroxy-6-methoxy-3-methyl-1,2,3,4,4a,9a-hexahydro-9H,10H-anthracen-9-one (8-hydroxytetrahydroaltersolanol B) of formula (8)

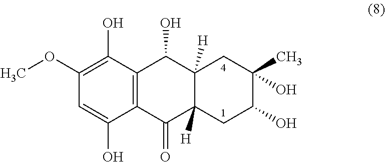

as an anti-infective against MDR pathogens. The structure of this compound has already been known, as well. As far as its acitivity is concerned, however, Xu et al., J. Antibiot. 61(7), 415-419 (2008) and Sommart et al., Chemical & Pharmaceutical Bulletin 56(12), 1687-1690 (2008) have only disclosed a slight effectiveness against Staphylococcus aureus or methicillin-resistant Staphylococcus aureus. The inventors, however, have found out that compound (8) indeed shows a very good activity against methicillin-resistant Staphylococcus aureus and even an excellent effectiveness against Streptococcus pneumoniae.

Consequently, the above compounds of formulae (4) to (7) will preferably be used against multiply drug resistant strains of methicillin-resistant Staphylococcus aureus (MRSA), Staphylococcus epidermis, Streptococcus pneumoniae, Enterococcus faecalis or faecium, Escherichia coli, Klebsiella sp., Pseudomonas aeruginosa, Aspergillus sp., as well as against respiratory viruses from the group of human rhinoviruses and respiratory syncytial viruses, and the compound of formula (8) will preferably be used against the same pathogens, except for Staphylococci, i.e. against Streptococcus pneumonia, Acinetobacter baumanii, Enterococcus faecalis or faecium, E. coli, Klebsiella sp., Pseudomonas aeruginosa, Aspergillus sp., as well as against respiratory viruses from the group of human rhinoviruses and respiratory syncytial viruses, which will be proved in detail by exemplary embodiments below.

The present invention also relates to a method for producing compounds according to the formulae (4) to (8), said method consisting in fermenting a microorganism producing the compound or one of its precursors under growth conditions and obtaining the respective compound from the culture, optionally after disrupting the cells of the microorganism in order to increase the yield. Such a fermentation method preferably uses a pure strain of Stemphylium globuliferum or Nigrospora sp., as the inventors could obtain the highest yields using these species. Alternatively, any other microorganism capable of producing the new compounds or their precursors, e.g. Alternaria sp., may be used for fermentation. If the fermentation yields precursors, said precursors may be converted into the desired new compounds of formulae (4) to (8) by any methods known to those skilled in the art of organic synthesis, optionally carrying out some steps of the method under the influence of enzymatic catalysis, in order to obtain higher enantioselectivities.

For example, it is possible to cultivate Alternaria sp. in order to obtain altersolanol B, or another member of the altersolanol family from the fermentation broth, which compound may be converted into a compound of any of the formulae (4) to (6) or (8), e.g. tetrahydroaltersolanol B, or a stereoisomer thereof by hydrating the non-aromatic double bond between the carbon atoms 4a and 9a and that of the keto group in position 9. Another synthesis pathway consists in cleaving an OH group together with the adjacent hydrogen atom, i.e. by dehydration, for example at the hydrated ring of altersolanol A or B, in order to obtain a double bond between the corresponding C-atoms as well as two prochiral centers in these positions. Subsequently, it is possible to enzymatically rehydrate or cis- or trans-dihydroxylate these double bonds, which will yield the desired compound if the stereospecifity of the enzyme (such as hydratase, peroxygenase) or oxidating agent (such as permanganate) is suitably selected. The alterporriols G and H may, for example, be obtained by chemically (again, for example, enzymatically) linking the corresponding anthraquinone substituent to position 8 of altersolanol M, i.e. the corresponding “monomeric” anthraquinone which is acetylated in position 3, which may optionally have been derivatized before, where-after the acetyl group is hydrolytically cleaved from the OH group in position 3.

Suitable enzymes for the synthesis steps for converting the precursors of the desired compounds may, in some cases, also be isolated from the microorganism used for fermentation or from another microorganism. In the latter case, it may be useful when, for example, a microorganism produces the desired product, but only in minor amounts or in a contaminated form so that, from an economic point of view, it is better to obtain a precursor of the product by cultivating another strain (or even another species or genus) and to use enzymatic synthesis in order to convert said precursor into the target compound.

Preferably, however, the respective compounds (4) to (8) are obtained by extraction and subsequent isolation from a crude extract of a culture, e.g. by fractional crystallization or chromatographic methods, more preferably by preparative HPLC, which, again, has resulted in the highest yields and highest purities so far. Of course, other methods of isolation are also contemplated, particularly in larger-scale experiments, e.g. by direct fractional crystallization of the crude extracts or by absorption procedures. However, one skilled in the art will be able to determine the best suitable isolation procedures for the respective cultivation (and/or synthesis or partial synthesis) steps without undue experimentation.

### EXAMPLES

Below, the invention will be described in further detail referring to specific exemplary embodiments, which, however, are not intended to limit the invention's scope in any way.

The compounds of the formulae (4) to (8) were obtained by cultivating Stemphylium globuliferum for the compounds (5) to (7) and Nigrospora sp. for the compounds (4) and (8), and by subsequent extraction.

### Isolation

a) Stemphylium globuliferum

Fresh, healthy stems of Mentha pulegium (pennyroyal) were used for isolating the endophytic fungus. The surface of the stems was sterilized using 70% ethanol for 1 min and then rinsed with sterile water in order to remove the alcohol. In order to distinguish any remaining epiphytic fungi from endophytic fungi, an imprint of the stem surface was obtained on organic malt extract agar. Small tissue samples from the interior were aseptically sectioned and pressed onto agar plates containing an antibiotic in order to suppress bacterial growth. The composition of the isolating medium was as follows: 15 g/1 malt extract, 15 g/l agar, and 0.2 g/l chloroamphenicol in distilled water, pH 7.4-7.8. A pure fungus strain was obtained from the cultures by repeated inoculation on malt extract agar plates. The fungus culture was identified as Stemphylium globuliferum according to a molecular biological protocol by means of DNA amplification and sequencing of the IST region. The sequence data was deposited with GenBank under access number EU859960.

### b) Nigrospora sp.

The isolation and identification were carried out in a way analogous to those of Stemphylium globuliferum, using leaves of Bruguiera sexangula, a mangrove species, instead of the pennyroyal stems.

### Cultivation

For cultivating the two isolated microorganisms, 1 liter Erlenmeyer flasks, each containing 100 g rice and 100 ml distilled water, were autoclaved in order to contain a swollen solid rice medium. A small part of the above isolating medium, containing the pure fungus strain, was applied to the solid rice medium under sterile conditions, and the fungus was cultivated at room temperature (22° C.) for 40 days in the case of Stemphylium globuliferum and for 30 days in the case of Nigrospora sp.

### Extraction and Isolation

After 30 and 40 days, respectively, the culture was extracted twice with 300 ml ethyl acetate (EtOAc). The EtOAc extraction was evaporated to dryness and partitioned between n-hexane and 90% aqueous methanol (MeOH). The aqueous-methanolic phase was evaporated to dryness, and the residue was subjected to column chromatography on a LH-20 column using 100% methanol (MeOH) as eluant, while being detected by means of a thin layer chromatography (TLC) on silica F245 (Merck, Darmstadt, Germany) using EtOAc/MeOH/H₂O (77:13:10) as eluant. The fractions containing the desired compounds were combined and subjected to semi-preparative HPLC (Merck, Hitachi L-7100) using a Eurosphere 100-10 C18 column (300×8 mm, L×i.d.) and a linear water-methanol gradient. This way, the compounds of the formulae (4) to (8) were obtained in their pure forms; it was, however, not possible to separate the atropisomers alterporriol G and H.

The proportions of the individual compounds in the overall yield were 49.0% by weight for alterporriol G and H, 10.4% by weight for altersolanol K, 7.8% by weight for altersolanol L, 14.6% by weight of tetrahydroaltersolanol B, and 18.2% by weight for 8-hydroxytetrahydroaltersolanol B.

The compounds were identified using their respective ¹H-NMR-, ¹³C-NMR-, HMBC-, and COSY-spectra as well as comparisons to the values quoted in the literature.

### Determination of Activity

The activities of the compounds were tested in two different screening systems. The antibacterial and antifungal activities were examined by means of an MIC test, MIC standing for “minimal inhibitory concentration” and referring to the lowest concentration of a substance at which no proliferation of microorganisms can be observed with the naked eye. The MIC is determined by means of a so called titration method in which the substance is diluted and, subsequently, the pathogen is added to it.

Usually, this method is applied in order to determine the concentration of an antibiotic which only just inhibits the growth of a bacterial strain. The MIC is indicated in micrograms per milliliter (μg/ml), and the dilutions are conventionally carried out in log2 steps. Herein, a starting concentration of 250 μg/ml was diluted several times, to the double volume in each case, resulting in test concentrations of 250 μg/ml, 125 μg/ml, 62.5 μg/ml, 31.2 μg/ml, 15.6 μg/ml, 7.8 μg/ml, etc. Thus, lower values indicate a better activity as an anti-infective.

The tests were carried out according to the standards of EUCAST (European Committee for Antimicrobial Susceptibility Testing) and according to the AFST-protocol (“Antifungal Susceptibility Testing” protocol) of the European Society of Clinical Microbiology and Infectious Diseases (ESCMID).

The screening system for viruses is an infection system which involves the infection of host cells in vitro, the test substance being added before or after the infection in order to determine its activity. All these tests were carried out according to the internal standard protocol for drug screenings of SeaLife Pharma, using analogous dilution series as in the antibacterial/antifungal test above.

In the following Table 3, the test results indicating the anti-infective action of altersolanol M, alterporriol I and J as well as of some known and structurally similar comparative substances against some multi-resistant bacteria and fungi (all of which were kindly placed at our disposal by Prof Georgopulos from the Medical University of Vienna). The data constituting averages of the values obtained in multiple test runs.

It becomes clear that the compounds of the formulae (4) to (8) show good to excellent activities against five bacterial species, i.e. against Enterococcus faecalis or faecium, methicillin-resistant Staphylococcus aureus, Streptococcus pneumoniae, Staphylococcus epidermis, and Acinetobacter baumanii, and, moreover, also show an activity against Aspergillus fumigatus or faecalis z, which is only moderate to low, though. In this series of tests, tetrahydroaltersolanol B was the only compound which was also weakly effective against E. coli, Klebsiella sp., and Pseudomonas aeruginosa.

As a result of testing the activity of the compounds (4) to (8) against three different species of respiratory viruses, that is against a human rhinovirus (hrv), a respiratory syncytial virus (rsv), and paraflu, all obtained from the ATCC, the inventors found out that, already at a concentration of 0.1 μg/ml, both (or all three) compounds provide the cells with 100% protection.

Thus, it was clearly shown that the use of the five known compounds according to the invention is sometimes highly efficient against both multiple drug resistant bacterial and fungal pathogens and respiratory viruses and that they may, thus, be used as anti-infectives in a wide range of applications.

TABLE 3
MHK values in multi-resistant bacteria and fungi

| Substances | EC | KL | PS | EK | MRSA | STR | SE | AB | ASP |
|---|---|---|---|---|---|---|---|---|---|
| Example 1: tetrahydroaltersolanol B (4) | 125 | 125 | 125 | | 31.2 | 15.6 | 31.2 | 31.2 | |
| Example 2: altersolanol K (5) | | | | 31.2 | 31.2 | 31.2 | 31.2 | | 62.5 |
| Example 3: altersolanol L (6) | | | | 7.8 | 7.8 | 7.8 | 7.8 | 7.8 | 31.2 |
| Example 4: alterporriol G + H (7) | | | | 7.8 | 7.8 | 7.8 | 7.8 | | 125 |
| Example 5: 8-hydroxytetra- | | | | 125 | 15.6 | 7.8 | 15.6 | | 31.2 |
| hydroaltersolanol B (8) | | | | | | | | | |
| Bostrycin | | | | | 125 | 62.5 | 125 | 125 | |
| Altenusin | 62.5 | | | 62.5 | 31.2 | 31.2 | 31.2 | 62.5 | |
| Alterporriol A + B | | | | 15.6 | 15.6 | 7.8 | 15.6 | 125 | 62.5 |
| Altersolanol A | 62.5 | | | 15.6 | 7.8 | 7.8 | 7.8 | 62.5 | 7.8 |
| Altersolanol J | | 125 | | 31.2 | 62.5 | 15.6 | 62.5 | 125 | 125 |
| Ampelanol | | | | | | | | | |
| Macrosporin | 62.5 | | | | 62.5 | | 62.5 | 125 | |
| Macrosporin sulfate | | | | | | 31.2 | | 125 | |
| Indole carbaldehyde (carboxaldehyde) | | | 125 | | | 62.5 | | 125 | |

EC E. coli
KL Klebsiella sp.
PS Pseudomonas aeruginosa
EK Enterococcus faecalis or faecium
MRSA methicillin-resistant Staphylococcus aureus
STR Streptococcus pneumoniae
SE Staphylococcus epidermis
AB Acinetobacter baumanii
ASP Aspergillus fumigatus or faecalis

1. A use of the following anthracene derivatives as anti-infectives:
a) (2R,3S,10R)-2,3,5,10-tetrahydroxy-6-methoxy-3-methyl-1,2,3,4,4a,9a-hexahydro-9H,10H-anthracen-9-one (tetrahydroaltersolanol B) 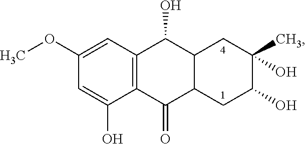
b) (1R,2R,3R,4aS,9aR,10R)-1,2,3,5,10-pentahydroxy-7-methoxy-2-methyl-1,2,3,4,4a,9a-hexahydro-9H,10H-anthracen-9-one (altersolanol K) 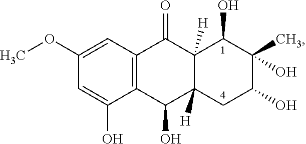
c) (2R,3R,4R,4aS,9aS,10R)-2,3,4,8,10-pentahydroxy-6-methoxy-3-methyl-1,2,3,4,4a,9a-hexahydro-9H,10H-anthracen-9-one (altersolanol L) 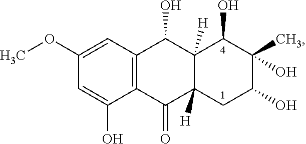
d) 8-(1,7-dihydroxy-6-methyl-3-methoxy-9,10-dioxo-9H,10H-anthracen-2-yl)-(1S,2S,3R,4S)-1,2,3,4,5-pentahydroxy-7-methoxy-2-methyl-1,2,3,4-tetrahydro-9H,10H-anthracen-9,10-dione (atropisomers alterporriol G and H)

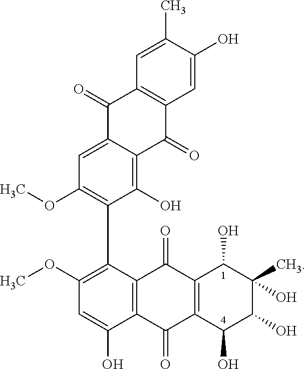

2. The use according to claim 1, characterized in that the anthracene derivatives are used as anti-infectives against multiply drug resistant (MDR) pathogens.

3. The use according to claim 2, characterized in that the compound is used against multiply drug resistant strains of methicillin-resistant Staphylococcus aureus (MRSA), Staphylococcus epidermis, Streptococcus pneumoniae, Acinetobacter baumanii, Enterococcus faecalis or faecium, Escherichia coli, Klebsiella sp., Pseudomonas aeruginosa, Aspergillus sp., as well as against respiratory viruses from the group of human rhinoviruses and respiratory syncytial viruses.

4. A use of (2R,3S,4aS,9aS,10R)-2,3,5,8,10-pentahydroxy-6-methoxy-3-methyl-1,2,3,4,4a,9a-hexahydro-9H,10H-anthracen-9-one (8-hydroxytetrahydroaltersolanol B) 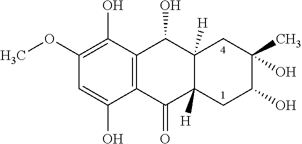
as an anti-infective against multiply drug resistant (MDR) pathogens selected from multiply drug resistant strains of Streptococcus pneumoniae, Acinetobacter baumanii, Enterococcus faecalis bzw. faecium, Escherichia coli, Klebsiella sp., Pseudomonas aeruginosa, Aspergillus sp., as well as against respiratory viruses from the group of human rhinoviruses and respiratory syncytial viruses.

5. A method for producing one of the compounds defined in any one of claims 1 to 4, characterized in that a microorganism producing the compound is fermented under growth conditions and the compound is obtained from the culture, optionally after having disrupted the cells of the microorganism.

6. The method according to claim 5, characterized in that Stemphylium globuliferum or Nigrospora sp. is used as the microorganism.

7. The method according to claim 5 or claim 6, characterized in that the compound is obtained by extraction and subsequent isolation from the crude extract.
